SEQUENCE LISTING (23 sequences)

SEQ ID NO: 1 - DNA, 21 bp, Artificial Sequence
  Description of Artificial Sequence CCR-5
ccttccaggaattctttggcc

SEQ ID NO: 2 - DNA, 21 bp, Artificial Sequence
  Description of Artificial Sequence Bac-CCR5
cctgtgggcaattctttgtga

SEQ ID NO: 3 - DNA, 21 bp, Artificial Sequence
  Description of Artificial Sequence Bac-CCR5
cctgtcggaaattctttagcc

SEQ ID NO: 4 - DNA, 21 bp, Artificial Sequence
  Description of Artificial Sequence Bac-CCR5
cctgtaggtaattctctagcc

SEQ ID NO: 5 - DNA, 21 bp, Artificial Sequence
  Description of Artificial Sequence Bac-CCR5
cctgttgggaattctctggcc

SEQ ID NO: 6 - DNA, 21 bp, Artificial Sequence
  Description of Artificial Sequence Bac-CCR5
ccttccaggaattctctcgcc

SEQ ID NO: 7 - DNA, 21 bp, Artificial Sequence
  Description of Artificial Sequence Bac-CCR5
ccttccaggaattctcttgcc

SEQ ID NO: 8 - DNA, 34 bp, Artificial Sequence
  Description of Artificial Sequence Bac-CCR5
ggaaatatctgtaggcctgtgacatctagaggtg

SEQ ID NO: 9 - DNA, 34 bp, Artificial Sequence
  Description of Artificial Sequence Bac-CCR5
cacctctagatgtcacaggcctacagatatttcc

SEQ ID NO: 10 - DNA, 40 bp, Artificial Sequence
  Description of Artificial Sequence CCR5 C-terminal fragment
aattcaggcctgcaccatcaccatcatcactaaggatcct

SEQ ID NO: 11 - DNA, 40 bp, Artificial Sequence
  Description of Artificial Sequence CCR5 C-terminal fragment
gtccggacgtggtagtggtagtagtgattcctaggagatc

SEQ ID NO: 12 - DNA, 21 bp, Artificial Sequence
  Description of Artificial Sequence FOR-CD4
cctaagctgatgctgagcttg

SEQ ID NO: 13 - DNA, 31 bp, Artificial Sequence
  Description of Artificial Sequence BAC-CD4
cagtggatccaatggggctgcaggtcttctg

SEQ ID NO: 14 - DNA, 33 bp, Artificial Sequence
  Description of Artificial Sequence CD4 C-terminal
gccccattcaccatcatcatcaccaccatttag

SEQ ID NO: 15 - DNA, 38 bp, Artificial Sequence
  Description of Artificial Sequence CCR5 C-terminal fragment
acgtcggggtaagtggtagtagtggtggtaattcctag

SEQ ID NO: 16 - DNA, 38 bp, Artificial Sequence
  Description of Artificial Sequence CD4-HIS3
gatccttaatggtggtgatgatggtgaatggggctgca

SEQ ID NO: 17 - DNA, 21 bp, Artificial Sequence
  Description of Artificial Sequence FOR-CD4
cctaagctgatgctgagcttg

SEQ ID NO: 18 - DNA, 31 bp, Artificial Sequence; identical to SEQ ID NO: 13
  Description of Artificial Sequence BAC-CD4

SEQ ID NO: 19 - DNA, 30 bp, Artificial Sequence
  Description of Artificial Sequence CD4-HIS5
gccccattcaccatcatcaccaccatttag

SEQ ID NO: 20 - DNA, 38 bp, Artificial Sequence; identical to SEQ ID NO: 16
  Description of Artificial Sequence CD4-HIS3

SEQ ID NO: 21 - DNA, 30 bp, Artificial Sequence; identical to SEQ ID NO: 19
  Description of Artificial Sequence CD4-HIS5

SEQ ID NO: 22 - DNA, 38 bp, Artificial Sequence; identical to SEQ ID NO: 15
  Description of Artificial Sequence CD4-HIS3

SEQ ID NO: 23 - DNA, 3369 bp, Homo sapiens
ctcatctggccagaagagctgagacatccgttcccctacaagaaactctccccgggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttgggttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatagaggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctgtctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttctaaaataa

(19) United States
(12) Patent Application Publication
(10) Pub. No.: US 2010/0098723 A1
(43) Pub. Date: Apr. 22, 2010

(54) VACCINE COMPOSITIONS AGAINST INFECTIOUS PATHOLOGIES FOR MAMMALS

(75) Inventor: Francisco VEAS, Mauguio (FR)

Correspondence Address: NIXON & VANDERHYE, PC, 901 NORTH GLEBE ROAD, 11TH FLOOR, ARLINGTON, VA, 22203, US

(73) Assignee: IRD, Paris (FR)

(21) Appl. No.: 12/418,897

(22) Filed: Apr. 6, 2009

Related U.S. Application Data
(63) Continuation of application No. 10/642,763, filed on Aug. 19, 2003, now abandoned.
Application No. 10/642,763 is a continuation of application No. 09/913,525, filed on Sep. 12, 2001, now abandoned.

(30) Foreign Application Priority Data
Feb. 15, 1999 (FR) 99 01 794

(51) Int. Cl. A61K 39/21; C07K 16/10; A61P 31/18

(52) U.S. Cl. 424/208.1; 530/387.9

(57) ABSTRACT

The invention concerns immunogenic compositions prepared from preparations obtained by incubating first means expressing the target receptor(s) of an infectious pathogenic agent, with second means expressing at least the regions of the infectious pathogenic agent recognising said targets, in conditions enabling interaction between the first and second means so as to form a complex, the incubation being carried out according to different time intervals, and contacting the resulting complexes with a binding agent, for different time intervals, said first and second means being tolerated by mammals. The invention is useful for preparing vaccine compositions designed for mammals against an infectious pathology.

The present application is a continuation of U.S. application Ser. No. 10/642,763, filed Aug. 19, 2003 (pending) (published as US 2004-0076640 A1) which is a continuation of U.S. application Ser. No. 09/913,525, filed Sep. 12, 2001, which is a 371 U.S. national phase of PCT/FR00/00375, filed 15 Feb. 2000, which designated the U.S., and claims benefit of FR9901794 filed Feb. 15, 1999, the entire contents of each of which are hereby incorporated by reference.

The purpose of the invention is immunogenic compositions that can be used particularly as vaccines against infectious pathologies for mammals.

The target pathologies according to the invention are of the dependent cell type, in other words the infectious process develops after the pathogenic agent has bonded (possibly but not necessarily followed by fusion) to a target mammal cell.

The important role played by some regions of these pathogenic agents in the infections that they cause, is well known.

Thus for example, various work on HIV has shown that the regions involved in interactions with target cell receptors (CD4 receptor and chemiokinetic receptors such as CCR5 and CXCR4) are preserved regions of the virus envelope.

In the past, the formation of antibodies against these regions has been hindered by the problem of access to interesting epitopes. Complex interactions and structural changes are involved during bonding of the virus to the target cell, followed by fusion, that actually prevents access to epitopes in fused regions, making it difficult to access epitopes in regions adjacent to regions directly involved in the interaction with the target cells, which in any case may be exposed, for only a short period.

With an experimental system described in Science, Vol. 283, Jan. 15, 1999, LaCasse et al. have probably achieved the formation of antibodies neutralizing HIV infectious isolates in mice, therefore achieving some access to the said epitopes.

The proposed system is obtained by fusion of simian fibroblasts modified to express the functional envelope of a primary isolate of HIV-1 (P168) with human neuroblastomas expressing the CD4 receptor and the CCR5 co-receptor, then binding of the complex formed 4 to 5 hours after the beginning of fusion, with formaldehyde. In tests on mice, it was demonstrated that a complex of this type is capable of forming antibodies neutralising infectious HIV isolates originating from a very wide variety of sub-types.

However, this type of system cannot be used in man due to the potential danger of cellular populations forming the immunogenic complex.

Furthermore, the conditions used to generate the system cannot guarantee that conformation of epitopes can be preserved or that access to a wide region of interest is possible.

The inventors have obtained complexes at a stage in which the epitopes of interest (including epitopes close to those involved in fusion) were exposed in a particularly satisfactory manner and must be preserved in their natural conformation, by controlling progress with fusion and the conditions under which binding is achieved. The immunogenic properties demonstrated in this type of complex then make them ideal candidates for generating vaccines, with the very significant advantage that they can be used in man when appropriate products are used for the preparation of complexes.

Advantageously, this type of system is generally applicable for any type of pathogenic agent with an infectious process that involves a bonding step, possibly but not necessarily followed by fusion with the target cells.

Therefore, the purpose of the invention is to supply immunogenic compositions and vaccinal compositions that can be used in mammals, against infectious pathologies dependent on cellular infection.

It is also intended for use with immunogenic complexes used in these compositions, as new products, and the antibodies formed against epitopes in the regions of the pathogenic agent nearby and even in the immediate vicinity of the regions involved in fusion with the target cell.

Immunogenic compositions according to the invention are characterized in that they are created from preparations obtained by:
incubation of first means expressing the target receptor(s) of an infectious pathogenic agent like that defined above, with second means expressing at least the regions of the pathogenic agent recognizing the said targets under conditions enabling interaction of the first and second means so as to form a complex, this incubation step being done with different intervals in order to produce complexes corresponding to different fusion stages and therefore with different exposures and conformation of newly demasked epitopes, and
putting complexes formed into contact with a binding agent for different intervals, in order to fix complexes with different exposures and conformations of epitopes against which antibodies are to be formed,

the said first and second means being chosen among products tolerated by mammals.

According to one embodiment of the invention, the said first means are mammal autologous cells. These are healthy cells originating from the mammal to be vaccinated, and for example from man, particularly total PBMCs, lymphocytes or isolated macrophages.

These cells are stimulated if necessary so as to express a sufficient quantity of the receptor(s) necessary for the required interaction.

In another embodiment of the invention, the said first means are vectors expressing the target receptor(s) at their surface. For example, these vectors comprise viral vectors such as baculovirus, the Semliki forest virus (SFV) and yeast such as Saccharomyces cerevisae.

According to another embodiment of the invention, the said first means are liposomes that have correctly presented target receptor(s) on their surface, thus mimicking target cells.

The target transmembrane receptors (with one or several passages) will be included in liposomes starting from receptor(s) expressed in large quantities, due to expression vectors, at the surface of the cells (10⁶ to 10⁷/cell) of either an insect, yeast or mammal. The cell receptors pass to liposomes after isolation of cellular membranes, and they are treated with an appropriate detergent using the protocols defined by J. L. Rigaud (see reference below) and finally their inclusion in appropriate membranes.

According to the invention, the second means used are means expressing at least the regions of the infectious pathogenic agent capable of bonding to the target cells and fusion with these cells.

Second means used according to the invention are thus composed of cells previously transformed with a vector carrying at least one bonding region with at least one target receptor. Advantageously, they are viral vectors such as those mentioned above, namely baculovirus, SFV, or yeast such as Saccharomyces cerevisae.

As a variant, the second means are composed of the viral vectors themselves.

In another variant, the second means are infected cells producing pathogenic agents or are composed of the infectious pathogenic agents themselves.

The pathogenic agents mentioned above may be viruses, and particularly retroviruses, bacteria, mycobacteria, or parasites such as Plasmodium sp, Leishmania sp, Trypanosoma cruzi and Trypanosoma brucei.

The invention is particularly useful in the case of HIV, this term being used in the description and the claims to denote isolates of various human or animal stocks, and viruses with a natural or recombining envelope, possibly muted.

The invention is thus aimed at immunogenic compositions in which the said preparations are obtained by incubation of the first means expressing the CD4 receptor and/or HIV co-receptors, with second means expressing at least preserved regions of gp120 or gp160 envelope proteins.

The expression “envelope protein” as used in the description and the claims encompasses the natural protein and also recombining proteins like those known to the expert in the subject, and muted proteins. Muted proteins have the advantage that they enable the identification of envelope sites involved in recognition by antibodies, as they are produced after fusion with target receptors.

In this type of composition, the first means used are composed of autologous cells of mammals as described above. These cells are stimulated so as to express the CD4 receptor and/or CD4 co-receptors such as CCR5, CXCR4, the band 3 protein or other transmembrane proteins, in sufficient quantities for the required interaction. For example, stimulation is made with PHA and/or IL2.

As a variant, the said first means are viral vectors expressing CD4 and/or HIV co-receptors at their surface. As described above, these vectors include baculovirus, SFV, and yeasts such as Saccharomyces cerevisae.

In yet another variant, the said first means are liposomes expressing the CD4 receptor and/or co-receptors as mentioned above, at their surface. These liposomes can thus comprise the CD4 receptor and/or HIV co-receptors.

In this type of composition advantageously including one of the first means mentioned above, the second means used express at least the preserved regions of the gp120 or gp160 envelope proteins.

These second means are composed of cells that had previously been transformed with an HIV viral envelope vector, or at least preserved regions of the gp120 or gp160 proteins. Advantageously, there are viral vectors as envisaged above.

As a variant, the second means consist of the viral vectors themselves.

In another variant, the second means are infected cells producing HIV or are composed of the HIV virus itself. Advantageously, a fusogenic virus originating from primary isolates is used.

Remember that according to the invention, the gp120 or gp160 proteins, or the proteins comprising at least the preserved regions of the gp120 or gp160 proteins, are in the natural form, or in a recombinant form, or in a mutated form.

Advantageously, the said second means are composed of these types of protein and therefore comprise a monomer soluble gp120, possibly in recombining form, or a gp120 or gp160 oligomer, possibly also in recombining form. There may also be parts of these proteins including at least the preserved regions.

In one advantageous embodiment of the invention, the second means comprise a monoclonal anti-co-receptor antibody. Examples of monoclonal antibodies are 17b, 48d and CG10. This arrangement makes it possible to access interesting epitopes near the site on which the co-receptor is bonded to gp120.

Preferred compositions of this type are in molecular form and comprise soluble monomer gp120 as the first means, soluble CD4 as the second means, and a monoclonal antibody as defined above or a fragment Fab of such an antibody.

The first and second means are incubated so as to form a complex in which the first means are engaged in a fusion process with the second means.

It is advantageous to carry out this step using different intervals, such that different fusion stages are possible, and the stage that gives the best results for the required immunogenic properties can be chosen by testing the animal.

Usually, intervals vary from 15 minutes to 5 hours.

Conformations of these various stages are fixed by the addition of an agent capable of stopping fusion without significantly denaturing the epitopes of interest.

One particularly attractive binding agent for this purpose is 2,2′-dithiopyridine (aldidrithiol-2 or AT-2 for short).

Other agents may be used, for example such as formol, particularly when aiming at research applications.

Binding is achieved with different intervals, so that the effect of the binding kinetics on immobilization of the presented epitopes can be studied.

The preparations obtained are recovered, washed and put into suspension in an appropriate buffer solution.

The next step is to evaluate the best fusion and binding times to induce the best immune response and to create antibodies that prevent infection.

The invention is aimed at new antigenic complex products resulting from fusion and binding steps.

These complexes are advantageously used to study and demonstrate interaction sites for gp120 and/or gp41 in the envelope, and also regions immediately adjacent to these sites, particularly when they are in crystalline form.

The preparation step for the immunogenic compositions defined above are also within the scope of the invention. This process includes the use of the said first and second means, and their fusion and binding as described above.

A study of the immunological properties of the compositions of the invention has demonstrated their high immunogenic power.

Thus, serums collected following the administration of these compositions to CD4+, CXCR5+ transgenic mice or rabbits has shown high contents of antibodies.

These serums, and the antibodies obtained from these serums, are then purified according to conventional techniques and are included in the invention.

These antibodies are thus characterized in that they are capable of recognizing an infectious pathogenic agent and thus inhibiting its infectious property, according to an antigen-antibody type reaction.

As demonstrated by experiments carried out in vivo on mammals, purified serums and antibodies are capable of inhibiting the infection capacity of a wide spectrum of primary HIV isolates.

Therefore, the invention is intended for vaccinal compositions characterized in that they contain an efficient quantity of immunogenic compositions as described above with an inert vehicle acceptable for administration to a mammal, in combination with an additive if necessary.

Examples of additives that could be used to increase the immunity reaction of the organism of the mammal to be vaccinated, include mineral additives such as aluminium phosphate, oily additives such as the incomplete Freund additive, bacterial additives such as the Freund complete additive. The Ribi additive is one particularly attractive additive.

The vaccinal composition is administrated by injection or orally, with a second dose three months after the injection.

Vaccinal compositions are administrated in a sufficient quantity and according to a protocol capable of making the host immune to antigens of the infectious pathogenic agent.

The following examples describe other characteristics and advantages of the invention:

### Preparation of a Cellular Immunogenic Composition

We will use firstly autologous human cells taken from the patient to be vaccinated and expressing CD4/CCRS and CXCR4 after stimulation for 3 to 6 days with PHA/IL2 using Riley et al.'s method as described in JVI 1998, 71, pages 8273-8280, and secondly a primary isolate of HIV-1 or cells infected by such an isolate.

These two populations are distributed in different Petri boxes for incubation at 37° C. for different times equal to 15, 30, 45, 60, 120, 180, 240 and 300 minutes respectively. For each experiment, 3×10⁶ to 10⁸ autologous cells are used, and for example the primary isolate 92 HT 593 or ACH168.10 derived from the Aids Research and Reference Reagent Program, NIH (EUA), which use the HIV CXCR4 and CCR5 co-receptors.

At the end of the planned interval, AT-2 will be added at a content of 600 to 1000 μm, in solutions in PBS. Binding is done at 4° C. for a time recommended by Rossio et al., JVI, 1998, 72, 7992, or 1 to 12 h. The cellular complexes formed are washed with PBS, recovered and put into suspension at a content of 10⁶ to 10⁸ cellular complex/0.1 ml in PBS/(DM50 10%) in order to keep them at −80° C.

The frozen immunogene is unfrozen and washed several times and then put in the presence of the same quantity of an additive, for example Ribi (R-700 or R-730).

Preparation of an Immunogenic Composition with Viral Vectors

The procedure described in example 1 is used, but using firstly a baculovirus system containing the CD4 and CCRS and/or CXCR4 receptors on the surface, and secondly the Vaccinia virus or the Semliki forest virus carrying the monomer gp120 or the gp120 and gp41 oligomer. Other experiments use a baculovirus system enabling the expression of proteins in its own membrane (Boublick et al., Biotechnology, 1995, 13:1079-84).

### Preparation of a Liposome-Based Immunogenic Composition

The procedure described in example 1 is used, but using liposomes carrying CD4 and CCRS and/or CXCR4 at their surface. These liposomes are prepared based on information given by Rigaud et al. in Biochem. Acta Phys. 1995, 1231:223-246 or Pitard et al. in Eur. J. Biochem. 1996, 235, 3769-3778, that describe liposomes carrying functional membrane or transmembrane proteins. They are put to incubate with HIV-1 or HIV-2 envelopes for fusion purposes. They are either HIV viruses with different recombining envelopes (use of pseudotyped complemented viruses) or viral vectors carrying recombining envelopes of HIV. Transmembrane receptors of cellular membranes are passed to liposomes using vectors with strong expression such as Vaccinia, baculovirus or Saccharomyces cerevisae capable of obtaining 10° to 10⁷ copies of receptors per cell.

### Vaccinal Preparation Administered to a Mammal Against an HIV-1 Infection

An immunogenic composition according to example 3 is used, after testing on an animal to evaluate its capability of producing an antibody response. This composition is washed several times in PBS, put into suspension in physiological serum, and then added to this Ribi composition as an additive.

The preparation is injected at a content of 0.05 ml to 1 ml to laboratory or experimental animals (mouse, monkey, man depending on the case). A repeat injection is made 4 to 6 months later.

### Cloning and Expression of the CCR5 Co-Receptor and/or the CD4 Receptor on the Surface of Insect Cells

### Cloning of CCR5/6Histidine and Expression of Baculovirus

1. CCR5 pcDNA3 contains a complete sequence of human CCR5 (Genbank access number NM 000579). PCR is used to amplify the C-terminal region between the Eco-RI site and TGA. The position of the EcoRI site starting from ATG (+10) is 793 and the position of the Stop codon is 1066.

2. This fragment is cloned in a pUC18 plasmide for sequencing, and the fragment is then re-inserted in CCR5 pcDNA3 in EcoRI-XbaI.

3. Thus, the CCR5 pcDNA3 plasmide is modified and becomes CCR5-6HIS pcDNA3.

4. The CCR5 gene plus its 6HIS tag in C-terminal is extracted from the pcDNA3 by cutting BamHI-BamHI.

5. The CCR5-6HIS gene is then added by BgI II-bgl I on the output side of the promoter p10 of the transfer vector (baculo) p119.

6. When the CCR5-6HIS gene has been added to the transfer vector p119, it is co-transfected with the DNA of the AcSLP10 virus in insect Sf9 cells (Autographa californica).

7. The expression of CCR5-6HIS is obtained at the surface of the cells. The density of these CCR5-6HIS is evaluated by scatchard (REF).

8. The functional characterization of this receptor is evaluated by the capacity of the HIV virus or HIV gp120 to bind to these cells.

### Cloning of CD4. A Similar Protocol is Used:

1. The 6 Histidine tag is added to pGEM-T plasmide containing the CD4 gene. The position of the Bsu361 site starting from the ATG (+1) is 1087 and the position of the Stop codon is 1375.

2. The CD4 gene with 6 Histadines added in C-terminal is added into the pGEMAc116T transfer vector by BgI II-bgI I, on the output side of the polyedrine promoter.

3. The pGEMAc116T CD4-6 HIS transfer vector will be co-transfected with DNA from the AcSLP10 virus into insect Sf9 cells (Autographa californica).

4. The expression of CD4-6 HIS is obtained at the surface of these cells. The density of these CCR5-6HIS is evaluated by scatchard (Cahoreau et al., 1992, Biochemistry, 74, 1053-1065).

5. The functional characterization of this receptor is evaluated by the capacity of the virus or HIV gp120 to bind to these cells.

### Expression of the CD4 and CCR5 Double Recombining Agent:

1. The CCR5-6 HIS and CD4-6 HIS genes are cloned in vectors p119 and pGEMAc116T respectively.

2. The expression of the CCR5-6HIS under the control of the p10 promoter and CD4-6 HIS is made under the control of the polyedrine promoter.

3. The two transfer vectors are co-transfected with DNA of the AcSLP10 virus into insect Sf9 cells (Autographa californica).

4. This produces Sf9 cells expressing a recombining pair CCR5-6HIS and CD4-6HIS in equal proportions, on their surface.

### Purification of Cellular Membranes Enriched in CCR5 or CD4 and Preparation of the Corresponding Proteoliposomes

Thus, it is possible to reconstitute receptors in liposomes containing the following, starting from Sf9 cellular membranes:

1. CCR5 only,

2. CD4 only,

3. CCR5 and CD4 in proportions chosen from cells expressing CCR5 and CD4 separately,

4. CCR5 and CD4 in chosen proportions starting from cells expressing CCR5 and CD4 at the same time, and in identical quantities.

The objective is to obtain HIV envelopes fusion with the HIV co-receptor and then to stop this fusion using a binding agent such as paraformaldehyde or glutaraldehyde and to inject this immunizing pair into huCD4/huCCR5 transgenic mice or into macaques or other monkeys. It may then be possible to inject the preparations into man, depending on the results.

The same system is set up for CXCR4.

### Strategies for the Reconstitution of Transmembrane Proteins in Proteoliposomes

SF9 cells of Autographa californica, that overexpress CCR5 (or CXCR4) and/or CD4 receptors, will be digested by appropriate detergents in order to obtain proteoliposomes using a method derived from Rigaud et al., 1988, Biochemistry, 27, 2677-2688, Paternostre et al., Biochemistry 1988, 27, 2668-2677; Gaymard et al., J. Biol. Chem. 1996, 271, 22863-22870.

Evaluation of the Functional Capacities of CCR5 and/or CD4

### 1—Expressed at the Surface of Sf9 Cells

a) Presence of receptors at the cellular surface analysed by FACS and confocal microscopy:

I. With specific anti-CD4 or anti-CCR5 antibodies,

II. With gp120 marked by specific antibodies,

III. With HIV-1 carrying muted or unmuted envelopes,

IV. Initially, the function of receptors on the surface of Sf9 cells is characterized and the number of molecules per cell for which we know the lipidic environment of cellular membranes, is quantified by scatchard (Cahoreau et al above).

b) Specific confocal fluorescence analysis of fusion by methods derived from Robert Blumenthal (NIH, personal communication, 2000) and by Vidal et al.'s methods, 1996, J. Biol. Chem. 270, 17823-17829.

I. After contact with cells expressing the HIV-1 envelope (Muted or not muted),

II. After contact with HIV-1 (or viral pseudotypes carrying muted or unmuted envelopes),

III. With viral pseudo-particles.

2—In the Corresponding Proteoliposomes

a) Specific confocal fluorescence analysis using the above methods

b) Other energy transfer methods (FRET: Fluorescence resonance energy transfer, Mattjus et al., 1999, Anal. Biochem. 268, 297-304).

### CCR-5, Introduction of 6 Histidine Residues in C-Terminal

### 1—Amplification by PCR of the C-Terminal Region Between the EcoRI Site and the TGA for CCR5:

| | |
|---|---|
| 5′ 3′ | |
| CCT TCC AGG AAT TCT TTG GCC | (SEQ ID NO: 1) |

Bac-CCR5: add a StuI site (created by degeneration of the genetic code) and an XbaI site into this oligonucleotide, for reintegration of the muted fragment into the original plasmide.

| | | | | |
|---|---|---|---|---|
| Val | gly | leu | opa | |
| GTG | GGC | TTG | TGA- | (SEQ ID NO: 2) |
| GTC | GGA | TTA | | (SEQ ID NO: 3) |
| GTA | GGT | CTA | | (SEQ ID NO: 4) |
| GTT | GGG | CTG | | (SEQ ID NO: 5) |
| | | CTC | | (SEQ ID NO: 6) |
| | | CTT | | (SEQ ID NO: 7) |

| | |
|---|---|
| StuI XbaI | |
| 5′ 3′ | |
| G GAA ATA TCT GTA GGC CTG TGA CAT CTA GAG GTG | (SEQ ID NO:8) |
| C CTT TAT AGA CAT CCG GAC ACT GTA GAT CTC CAG | (SEQ ID NO:9) |
| 3′ 5′ | |
| matched not matched | |

The amplified EcoRI-XbaI fragment is cloned in a pUC vector in EcoRI-XbaI and is then sequenced. The muted fragment is then reinserted in the original EcoRI-XbaI plasmide.

### 2—Introduction of the 6 Histidine Codons on the Output Side of the CCR5C-Terminal

The plasmide thus modified is cut by StuI and Xba and is then bonded with the StuI-XbaI DNA fragment described below. This fragment carries 6 Histidine codons and a Stop TAA codon.

```
    1/2 EcoRI StuI BamHi 1/2XbaI
AA TTC-A GGC CTG CAC-CAT-CAC-CAT-CAT-CAC TAA GGATCC T
  G T CCG GAC GTG-GTA-GTG-GTA-GTA-GTG ATT CCTAGG AGATC
(SEQ ID Nos: 10 and 11, respectively)
```

An Eco-RI site is added on the input side to clone oligonucleotides matched in an intermediate pUC vector, and thus to verify the sequence.

### Modification and Cloning of CD4

1—Sequencing of the C-Terminal Region of the pGEM-T Plasmide Containing the CD4 Gene:

The C-terminal region of the plasmide is verified by sequencing after a PCR* step.

### 2—Addition of 6 Histidine Residues in the CD4 C-Terminal:

1-Amplification of the Bsu361-Banim Region by PCR (in the Polylinker)

| | |
|---|---|
| PCR oligonucleotide: | |
| FOR-CD4: | |
| 5′ 3′ | |
| CCT AAGCTG ATG CTG AGC TTG | (SEQ ID NO: 12) |
| BAC-CD4: | |
| BamHi PstI | |
| 5′ 3′ | |
| CAGT GGATCC AAT GGG GCT GCA GGT CTT CTG | (SEQ ID NO: 13) |
| 2-Addition of 6 His codons | |
| 1/2 PstI 1/2 BamHI | |
| GC CCC ATT CAC CAT CAT CAT CAC CAC CAT TTA G | (SEQ ID NO: 14) |
| ACGTCG GGG TAA GTG GTA GTA GTG GTG GTA ATT CCTAG | (SEQ ID NO: 15) |
| PCR* type oligonucleotide | |
| CD4-HIS5 | |
| 5′ 3′ | |
| GCCCCATTCACCATCATCACCACCATTTAG | (SEQ ID NO: 21) |
| CD4-HIS3 | |
| 3′ 5′ | |
| ACGTCGGGGTAAGTGGTAGTAGTGGTGGTAATTCCTAG | (SEQ ID NO: 22) |
| 5′ 3′ | |
| GATCCTTAATGGTGGTGATGATGGTGAATGGGGCTGCA | (SEQ ID NO: 16) |
| a | |
| FOR-CD4 CCTAAGCTGATGCTGAGCTTG 40 | (SEQ ID NO: 17) |
| BAC-CD4 CAGTGGATCCAATGGGGCTGCAGGTCTTCTG 40 | (SEQ ID NO: 18) |
| CD4-HIS5 GCCCCATTCACCATCATCACCACGATTTAG 40 | (SEQ ID NO: 19) |
| CD4-HIS3 GATCCTTAATGGTGGTGATGATGGTGAATGGGGCTGCA 40 | (SEQ ID NO: 20) |

### Marking of Cells Expressing the HIV-I Envelope Using Calcein

### Reagents:

cellular culture
AM calcein (Molecular Probe, Inc) 1 μg/ml (−2.5 mM) (50 μg of calcein/20 μl of DMSO, stored at −20° C.)
DMSO (dimethyl sulfoxide)
RPMI or DMEM
PBS pH 7.4 (Dulb PBS by Gibco BRL)
centrifuge with IEC Central MOOR

### Method:

1. Count cells in the flask

2. Centrifuge at 1000 rpm for 5 minutes

3. Put cells back into suspension in 5 ml of the medium (for example 0.5×10⁶ cells/ml)

4. Addition of 1 μl of calcein/5 ml of cells (about 2.5 mmoles of calcein)

5. Vortex in order to obtain a good mix

6. Incubate for 45 minutes at 37° C., 5-7% of CO₂, sheltered from light

7. Centrifuge at 1000 rpm for 5 minutes

8. Wash twice with 10 ml of the medium (or PBS)

9. Put the cells back into suspension in 5 ml of the medium (or PBS) or at a concentration of 0.5×10⁶ cells/ml

10. Incubate for 30 minutes at 37° C., 5-7% of CO₂, sheltered from light

11. Wash twice with 10 ml of the medium (or PBS)

12. Put cells back into suspension in 5 ml of medium (or PBS) at a concentration of 0.5−1×10⁶ cells/ml Vortex

Marking of Target Cells with CMTMR

CMTMR is a fluorescent derivative of chloromethyl that diffuses freely through membranes of living cells. Once inside the cell, the slightly thiol-reactive probe is subject to what must be a reaction mediated by glutathione-S— transferase to produce fluorescent impermeable colored additives at the membrane.

The color of cells with CMTMR gives a bright fluorescence due to the reaction with proteins in the perinuclear regions, ER and Golgi, that are motionless, and lower fluorescence due to the fluorescent glutathion additive (PM about 600 Da) in cytosol that is capable of diffusing through small fusion pores.

1. The CMTMR cytoplasmic fluorescent probe (5-(and -6-)-(((4-chloromethyl), benzoyl) amino) tetramethylrhodamine) is solubilized (Molecular Probes cat #C-2927, ex/em 541/565 nm) at a concentration of 10 mM in DMSO.

10 or 20 μl samples of aliquot are taken and stored at −20° C. A dilution of CMTMR (1:500) in DMEM plus 10% of SVF deactivated by heat, 100 μ/ml of penicillin, 100 μg/ml of streptomycin (D10), is prepared.

2. The medium of target cells (on micro wells) is eliminated and 1 ml of a dilute solution of CMTMR is sprayed. The samples are incubated at 37° C. for 45-60 minutes. The coloring solution is replaced by 1 ml of D10 and incubation is continued for another 15 to 30 minutes.

### Carrying Out the Fusion

1. Effector cells marked by calcein (2 ml) are added to target cells marked by CMTMR. The two cellular populations are incubated for 3 to 5 h at 37° C.

2. At the end of the incubations, the medium is replaced by 1 ml of D-PBS and the phase and fluorescence images are taken with immersion lenses in 40× oil.

FITC is used to observe the cells colored with calcein (exciter BP 470-490; prism DM 505; transmitter BA 515-550) and rhodamine (exciter BP 530-550; prism DM 570; transmitter BA 590) is used for CMTMR. This avoids the overflow that occurs when coloring agent fluorescence is used. The images are brought together with 6 to 10 different filters chosen at random for each sample.

3. The data are analysed using the Metamorph software (Universal Imaging Inc.), superposing and counting images. The total number of positive cells is counted for CMTMR, and the total number of positive cells is counted for the two fluorescent probes. Bright field images are used to distinguish positive contents for which marked cells overlap each other, but are not fused. The fusion percentage is calculated as follows:

% fusion=[100×number of positive cells for 2 coloring agents][total number of target cells]

1. An immunogenic composition, wherein the composition is created from preparations obtained by:
(a) incubation of first cells expressing target receptor(s) of an infectious pathogenic agent, causing infections in a mammal by bonding and then fusion with target cells, with second cells expressing at least regions of the infectious pathogenic agent recognizing the said target receptor(s) under conditions enabling interaction of said first cells and said second cells so as to form a complex, this incubation step being done with different intervals in order to produce complexes corresponding to different fusion stages, and
(b) putting the complexes formed into contact with a binding agent for different intervals, in order to bind complexes with different exposures and conformations of epitopes against which antibodies are to be formed, the said first cells and said second cells being tolerated by mammals.

2. The composition of claim 1, wherein said first cells are autologous mammalian cells.

3. The composition of claim 1, wherein said first cells are transformed with vectors comprising genes expressing the target receptor(s).

4. The composition of claim 1, wherein said second cells are transformed with vectors carrying at least one bonding region to at least one receptor.

5. The composition of claim 1, wherein said second cells are transformed with viral vectors carrying at least one bonding region to a target receptor.

6. The composition of claim 1, wherein said second cells are infected cells that produce infectious pathogenic agents or are composed of infectious pathogenic agents.

7. The composition of claim 1, wherein said infectious pathogenic agent is a virus.

8. The composition of claim 7, wherein said pathogenic agent is HIV.

9. The composition of claim 1, wherein said preparations are obtained by incubation of first cells expressing the CD4 receptor and/or HIV co-receptors with second cells expressing at least the preserved regions in gp120 or gp160 envelope proteins.

10. The composition of claim 9, wherein said first cells are-autologous mammalian cells stimulated so as to express the CD4 receptor and/or HIV co-receptors, in a sufficient quantity for the required interaction.

11. The composition of claim 9, wherein said first cells are transformed with viral vectors comprising genes expressing CD4 and/or HIV co-receptors.

12. The composition of claim 9, wherein said second cells are transformed with viral vectors comprising at least regions of HIV-1 gp120 or HIV-1 gp160 envelope proteins.

13. The composition of claim 9, wherein said second cells are infected cells producing HIV or are composed of the HIV virus itself.

14. The composition of claim 9, wherein said second cells express HIV-1 gp120 or HIV-1 gp160 envelope proteins in natural or recombining form.

15. The composition of claim 9, wherein one of the co-receptors of HIV is replaced by a monoclonal antibody directed to a region of gp120 which binds to HIV-1 co-receptors.

16. The composition of claim 1, wherein said preparations are fixed with aldidrithiol-2 after incubation.

17. An isolated serum or antibody formed against the composition of claim 1.

18. The composition of claim 1, further comprising an inert vehicle acceptable for administration to a mammal, and optionally with an additive.

19. The composition of claim 3, wherein said vectors are viral vectors.

20. The composition of claim 6, wherein said infectious pathogenic agents are selected from the group consisting of a retrovirus, a bacteria, a mycobacteria, and a parasite.

21. The composition of claim 6, wherein said infectious pathogenic agents are selected from the group consisting of a Plasmodium sp., a Leishmania sp., Trypanosoma cruzi and Trypanosoma brucei.

22. The composition of claim 2, wherein said first cells are healthy human cells taken from a patient to be vaccinated.

23. The composition of claim 11, wherein the viral vectors are baculoviruses or Semliki forest viruses.

24. The composition of claim 9, wherein said first cells are yeast expressing CD4 and/or HIV co-receptors at their surface.

25. The composition of claim 24, wherein said yeast are Saccharomyces cerevisiae.

26. An immunogenic composition for forming serum or antibody recognizing an infectious pathogenic agent, wherein the composition is created from preparations obtained by:
(a) incubation of first cells expressing target receptor(s) of an infectious pathogenic agent, causing infections in a mammal by bonding and then fusion with target cells, with second cells expressing at least regions of the infectious pathogenic agent recognizing the said target receptor(s) under conditions enabling interaction of said first cells and said second cells so as to form a complex, this incubation step being done with different intervals in order to produce complexes corresponding to different fusion stages, and
(b) putting the complexes formed into contact with a binding agent for different intervals, in order to bind complexes with different exposures and conformations of epitopes against which antibodies are to be formed, the said first cells and said second cells being tolerated by mammals.

27. An isolated serum or antibody for recognition of an infectious pathogenic agent, wherein said serum or antibody is formed against the composition of claim 26.
